(19) United States
(12) Patent Application Publication
(10) Pub. No.: US 2003/0078204 A1
(43) Pub. Date: Apr. 24, 2003

(54) Recombinant production of human histone 1 subtypes and their use for therapeutic purposes

(76) Inventors: Kai Pohlmeyer, Fitzbek (DE); Bert Behnke, Ahrensburg (DE); Ralf Zabiensky geb. Wick, Hamburg (DE); Gerd Mayer, Hamburg (DE)

Correspondence Address: SCHWEGMAN, LUNDBERG, WOESSNER & KLUTH, P.A., P.O. BOX 2938, MINNEAPOLIS, MN, 55402, US

(21) Appl. No.: 10/194,405

(22) Filed: Jul. 12, 2002

Related U.S. Application Data
(63) Continuation of application No. PCT/EP01/00290, filed on Jan. 11, 2001.

(30) Foreign Application Priority Data
Jan. 13, 2000 (DE) 100 01 113.6

(51) Int. Cl. A61K 38/17; C12P 21/02; C12N 1/21; C07H 21/04; C12N 9/22; C12N 15/74

(52) U.S. Cl. 514/12; 435/69.1; 435/320.1; 435/252.33; 530/358; 536/23.2; 435/199

(57) ABSTRACT

The invention relates to recombinantly produced human histone-1 subtypes and to their use for therapeutic purposes.

The invention relates to recombinantly produced human histone 1 subtypes and their use for therapeutic purposes.

The protein histone 1 and its various subtypes, such as, for example, the histone 1° and the histone 1.2 as well as other histones together with DNA are the main components of chromatin. They play an important role in the organisation of the chromatin structure and in gene repression.

It is also known that the histones have functions outside the cell nucleus or the cell. Thus, lymphocytes of the spleen and thymus can secrete histones into the circulatory system in the course of apoptosis (D. A. Bell et al (1990), J. Clin. Invest. 85, 1487-1496). Furthermore, in sea urchins H1 histones are integral components of the cytoplasm (Multigner, L., Gagnon, J., Van Dorsselaer, A., Job, D. (1992) Nature 360, 33-39). It has also been reported that extracellularly occurring histone H1 can act as a receptor for thyroglobin, a hormonally active glycoprotein from the thyroid, on the plasma membrane of mouse macrophages (Brix, K., Summa, W., Lottspeich, F., Herzog, V. (1998) J. Clin. Invest. 102, 283-293). Cell-surface histones are responsible for the formation of auto-antibodies in autoimmune diseases such as SLE syndrome (systematic lupus erythematosus) (Holers, V. M., and Kotzin, B. L. (1985) J. Clin. Invest.76, 991-998).

It is also known that histones can exhibit antimicrobial effects not only in vitro (Hirsch, J. D. (1958) J. Exp. Med. 108, 925-944), but also in the human gastro-intestinal tract (Rose, F. R. A. J., Bailey, K., Keyte, J. W., Chan, W. C., Greenwood, D., Mahida, Y. R. (1998) Infection and Immunity 66, 3255-3263) or on the skin of fish species (Robinette, d., Wada, S., Arroll, T., Levy, M. G., Miller, W. L. Noga, E. J. (1998) Cell. Mil. Life Sci. 54, 467-475). This antimicrobial activity of histones is due to the presence of N-terminal peptides having antimicrobial action such as buforin 1 represented by the amino-terminal 39 amino acids of the histone H2A (Kim, H. S., Park, C. B., Kim, M. S., Kim, S. C. (1996) Biochem. Biophys. Res. Commun. 10, 940-948), or parasin, which is also an N-terminal cleavage product of the histone H2A.

The histones have acquired substantial importance for therapeutic applications through their action as cytostatics for eukaryotic cells. Thus, it is known that the viability of various leukaemia cell lines both in vitro and also tumour growth in vivo can be suppressed by purified bovine histone 1 (Class, R., Lindman, S., Fassbender, C., Leinenbach, H.-P., Rawer, S., Emrich, J. G., Grady, L. W., Zeppezauer, M. (1996) Am. J.Clin. Oncol. 19(5), 522-531). According to this publication, however, the purified bovine histone 1 does not act toxically on non-transformed PBMC (peripheral blood mononuclear cells). This suggests that the PBMC can only be attacked by histones after transformation into tumour cells.

The purification of mouse H1 histones expressed in E. coli is described in Biotechnol. Appl. Biochem. (1997, 26, 117-123). Expression of human H1 histones is not discussed there.

The heterologous expression of human H1 histones in yeast is described by W. Albig et al. in FEBS Letters (1998), 435, 245-250; C. Saunders und R. S. Cohen reported the expression of human histone H4 in E. coli (Biotechnics 1999, 26, 30-34).

The use of pure histone H1 for therapeutic purposes, especially as a cytostatic, is disclosed in the European Patent EP-B1-0 392 315. According to this document, the H1 histones used were isolated from bovine thymus cells.

It can be taken as a starting point that human histones are better suited to therapeutic application in humans than bovine or mouse histones, for example.

It is also advantageous if these histones are prepared by recombinant methods of production since, in view of the quantities required for therapeutic application, these methods are substantially more efficient and favourably priced than, for example, the expensive isolation of histones from the human or bovine thymus.

Regardless of the fact that recombinant production substantially facilitates the validation and monitoring of the production process, production in genetically modified organisms offers important advantages in respect of virus safety and in-process controls.

Consequently, a substantial object of the invention is to prepare human histone types or subtypes which can be produced recombinantly by an efficient biotechnological method.

In addition, an important object of the invention is to demonstrate the possibilities for the therapeutic usage of histone types according to the invention.

It is especially the object of the present invention to prepare human histone subtypes which exhibit high cytotoxicity to tumour cell lines at low dose.

Further objects are obtained from the following description.

These objects are achieved by the subject matter of the independent claims, especially based on the provision of the histone subtypes H1° and H1.2 according to the invention.

Advantageous embodiments are described in the dependent claims.

The present invention thus relates to proteins having the biological activity of human histone 1 or an active fragment thereof, produced recombinantly in prokaryotic cells, especially in E. coli. In connection with the invention, a biologically active fragment means that the imparted biological activity is sufficient for a therapeutic application. The invention especially relates to recombinantly produced human histone 1 proteins of the subtype H1° or H1.2.

According to the invention, the human H1 proteins are produced by a method which comprises the following steps:

a) Expression of a nucleic acid sequence coding for a human protein having the biological activity of a histone 1 or an active fragment thereof in prokaryotic cells, especially E. coli and

b) Extraction and purification of the recombinant human protein having the biological activity of a histone 1 or the active fragment thereof from the prokaryotic cells, especially E. coli.

It should be mentioned that in view of the strong anti-microbial action of the histones, it should be seen as extremely surprising that any expression at all and especially an efficient expression of histones is possible in bacteria. This result could not be expected from a knowledge of the prior art, see for example, Hirsch, J. D., 1958, vide supra.

A further object of the invention is to provide uses of the human histones H1 according to the invention, especially H1° and H1.2, for therapeutic purposes.

The subject matter of the invention is especially the use of the histones according to the invention for cancer therapy. This, for example, includes the use of histones according to the invention for the therapy of carcinomas, melanomas, sarcomas, mesotheliomas and malignant diseases, especially of the lymphatic system which are caused by malignant B and T cells, such as B-lymphoblastic lymphoma, myelogenic lymphoma or Burkitt's lymphoma.

histone 1 isolated and purified from cattle was used as a suitable reference sample to study the cytostatic effects of the human histones H1° and H1.2 according to the invention since the histones are highly conserved molecules. For example, human histone H4 and bovine histone H4 show more than 90% homology. Mouse histone H1.2 and human histone H1.2 show more than 97% homology and more than 88% identity. Unfortunately, no corresponding data are available for the bovine H1° and H1.2 sequences.

Two surprising observations were made during the use according to the invention of the human histones H1° and H1.2 according to the invention for the treatment of tumour cell lines:

Firstly, it was found that the human recombinant histones H1° and H1.2 according to the invention as well as the conventionally obtained bovine histone 1 have cytotoxic effects on healthy PBMCs which differ from the effects described hitherto. Thus, for example, it was reported in the prior art that purified bovine histone 1 indeed has a cytotoxic effect on a series of leukaemia cell lines but has no effects on PBMCs not transformed into tumour cells (Class, R. et al., vide supra). In contrast thereto, it has now been found that the human recombinant histones H1° and H1.2 according to the invention as well as the bovine histone 1 exhibit and time- and dose-dependent cytotoxicity both on leukaemia cell lines and on PBMCs.

Whereas the toxicity to the leukaemia cells appears directly after supplying the histones according to the invention, significant toxicity to PBMCs is only observable after a time period of at least one hour. After incubation for 24 hours, however, the recombinant human histones H1° and H1.2 as well as the reference sample of bovine histone 1 show a toxicity higher than 50% at a dose of 250 μg/ml.

The recombinant human histone H1.2 according to the invention shows cytotoxic effects on tumour cells, especially on leukaemia cell lines, which are similar to those of bovine H1 histone in terms of dose and time dependence.

Whereas the cytotoxic effects of human H1° on PBMCs are similar to those of the human histone 1.2 and also to the reference sample of bovine histone H1 in terms of dose and time dependence, it was now surprisingly found as a further substantial aspect of the invention that the human recombinant H1° according to the invention is less toxic to tumour cells, especially to leukaemia cells, than the human recombinant H1.2 according to the invention and the bovine histone 1. Such a difference in the cytotoxic effects of histone subtypes of a histone had not been described hitherto and was not to be expected in view of the sequence homology of the human histone subtypes H1° and H1.2.

In view of the time-dependent toxicity of the histones to PBMCs of healthy donors described above for the first time, for the use of human histone 1 for the treatment of tumours it is particularly important to use human histone 1 subtypes which have a toxic effect on malignant cells even at low dose. It had not yet been known hitherto that different histone subtypes of a histone can differ in terms of their cytotoxicity. Thus, for the first time a histone 1 subtype is provided by the present invention with the human histone H1.2 according to the invention, which is especially advantageously suited to use for cancer therapy as a result of its cytotoxicity properties and its human sequence.

Without wishing to be bound to a hypothesis, the following explanation is currently accepted for the different toxicities of the histones H1° and H1.2 to tumour cell lines: it was shown that histones and other basic proteins can form conducting channels in various membranes (Kleine, T. J., Lewis, P. N., Lewis, S. A. (1997) Am. J. Physiol. 273, C1925-C1936; Gamberucci, A., Fulceri, R., Marcolongo, P., Pralong, W. F., Benedetti, A. (1996) Biochem. J. 331, 623-630; Kleine, T. J., Gladfelter, A., Lewis, P. N., Lewis, S. A. (1995) Am. J. Physiol. 268, C1114-C1125). As a result of this channel formation, the permeability of the cell membrane for small monovalent cations and anions is increased and it is assumed that this increased permeability leads to cell tumefaction and ultimately to cell lysis.

It may be possible that this increased channel activity is a consequence of increased stability of the histone binding to the negatively charged phospholipid membrane. However, since the histones H1° and H1.2 have a very similar total charge (H1°: 61 amino acids positively charged, 7 amino acids negatively charged; H1.2: 62 amino acids positively charged, 7 amino acids negatively charged), it seems improbable that the stronger cytotoxicity of the histone H1.2 is based on a higher affinity for the negatively charged cell membrane.

Before channel formation takes place, recognition and binding to the membrane is necessary. It was proposed that this takes place through a histone H1 receptor on the membrane of the malignant cells. It is thus assumed that the varying cytotoxicity of H1.2 and H1° is based on the fact that a histone receptor with a stronger affinity for histone H1.2 is present on the cell surface. Assuming similar binding characteristics, the different toxicities could only be explained by different channel properties of H1° and H1.2.

The subject matter of the invention is also the use of the human histones H1 according to the invention as anti-microbial active substances, especially chemotherapeutic agents and antibiotics. The histones according to the invention are especially effective against a broad range of micro-organisms, including bacteria, virus, fungi and parasites.

The invention also relates to the use of histones according to the invention for the therapy of endocrine disorders. The histones according to the invention can also be used more advantageously for the therapy of immune diseases, especially for the therapy of auto-immune diseases such as, for example, SLE syndrome.

The present invention thus covers every possible form of the use of the human histones H1 produced recombinantly according to the invention in prokaryotic cells, especially E. coli, preferably the histones H1° and H1.2, especially preferably the histone H1.2, the biological activity of which brings about a therapeutic effect.

The invention further relates to the use of recombinant human histones H1 as carriers for nucleic acids, especially DNA molecules, RNA molecules, ribozymes, oligonucleotides and the like. In this case, the nucleic acids can act as active ingredients or as vaccines. As a result of their nucleus-targetting function, the histones according to the invention are particular well suited for this purpose. It is preferred that complexes are first allowed to form between the histones and the nucleic acid molecules and then the histone/nucleic acid complexes are applied as a combination.

As a result of their excellent properties as a carrier for nucleic acids, the histones according to the invention are especially well suited for the ex vivo treatment of cells with nucleic acids, especially with DNA. Thus, by using the histones according to the invention as an excipient/adjuvant in the transfection of cells, the transfection efficiency can be enhanced significantly.

The following examples serve to explain the invention without restricting the invention in any way.

### EXAMPLES

### Example 1

Isolation of the Histones H1° and H1.2 from E. coli

a) cDNA cloning and expression: cDNAs for the histones H1° and H1.2 were obtained from total RNA from HepG₂-cells (Aden et al. (1979) Nature 283:615-616; Knowles et al. (1980) Science 209:497-499) by RT-PCR. For this purpose HepG₂-cells were cultured in RPMI 1640 (Biochrom, Berlin) with 10% FCS (Biowhittaker, Verviers, Belgium). The total RNA was isolated with Trizol (Gibco BRL, Rockville, Md.) as recommended by the manufacturer. The reverse transcription was carried out using 2 μg of the total RNA and 500 ng oligo[dT30]-primer under conditions recommended by the manufacturer (Gibco BRL). 1 μl of the cDNA was used for the RT-PCR with the following primers:

H1° (forward):

5′ GGGGGGGGATCCATATGACCGAGAATTCCACGTCCGCCC 3′

H1° (reverse):

5′ GGGGGGGTCGACTCACTTCTTCTTGCCGGCCCTCTTGGC 3′

H1.2 (forward):

5′ GGGGGGGGATCCATATGTCCGAGACTGCTCCTGCCGC 3′

H1.2 (reverse):

5′ GGGGGGGTCGACCTATTTCTTCTTGGGCGCCGCCTTC 3′

The PCR comprised a 5-minute denaturation step at 94° C., followed by 35 cycles of one minute each at 94° C., one minute at 52° C. and one minute at 72° C. and a final elongation step of 10 minutes at 72° C.

The coding clone for H1° matched 100% with the sequence having Accession No. X03473 (D. Dönecke, R. Tonjes (1986), J. Mol. Biol., 187 (3), 461-464) and the coding clone for H1.2 matched 100% with the sequence having the Accession No. X57129 (S. Eick et al. (1989), Eur. J. Cell Biol. 49 (1), 110-115). The PCR products were cloned in the vector pET24 (a) (Novagen, Madison, USA) and checked by Sanger sequencing. Both clones were expressed in E. coli BL21 (DE3).

b) Extraction of H1° and H1.2 from E. coli: After induction for 2 hours with IPTG, the bacteria were harvested, lysed by pressure and centrifuged at 11,000×g. The supernatant was discarded and the histone-containing pellets were extracted with 0.75 M perchloric acid. The extractions were carried out for 15 minutes on ice, stirring continuously. The mixture was purified by centrifuging (11,000×g) and subsequent filtering through a 0.45 μm cellulose acetate membrane (Sartorius, Göttingen, Germany). The clear lysates were subjected directly to HPLC.

c) Reversed-phase chromatography: methanol was added to the acidic histone extracts up to a quantity of 10% of the final volume and the extracts were transferred to a reversed-phase C18 (5 μm)-column. After washing with five parts by volume of buffer A (0.1% trifluoroacetic acid, TFA) the H1 histones were separated by using an increasing acetonitrile water gradient. The H1° histone was usually eluted in the range of 20-35% acetonitrile whereas H1.2 was eluted at 25-45% acetonitrile. The purity of the histone fractions was determined by SDS-PAGE according to Lämmli and by analytical HPLC, which was carried out on a reversed-phase Vydac 218TP54 C18 column. The histones were frozen at −20° C. and lyophilised. For the cytotoxicity assays the histone preparations were diluted in sterile and endotoxin-free distilled water to a final concentration of 10 mg per ml. RPMI containing 10% FCS was used for further dilutions.

### Comparative Example 1

Extraction of H1 Histones from Bovine Thymus.

Frozen bovine thymus cells were homogenised at 4° C. in 50 mM Tris/HCl, pH 7.5; 5 mM MgCl₂, 5 mM DTT and 250 μm PMSF using a Waring mixer. After centrifuging at 1,500×g, the crude nuclear pellets were extracted using 0.75 M perchloric acid for 2 hours, stirring continuously (Pehrson, J. R. and Cole, R. D. (1981) Biochemistry 20, 2298-2301). The mixture was purified as described in example 1 and subjected directly to HPLC.

The fractions 3 to 6 obtained in the SDS-PAGE, which contained only two main bands in common with the 30 kDa standard, were pooled and designated as bovine histone H1 (see FIG. 1C). Analytical HPLC yielded two main peaks and a smaller peak (see FIG. 1C). The first peak represents histone 1° with a calculated content of 2.9% of this preparation. The second peak, which represents 13.5% of the proteins, could not be assigned to either H1° or H1.2. The main peak, representing 83.6% of the proteins, could be assigned to H1.2 because of its retention time. It is therefore assumed that H1.2 is responsible for the cytotoxic effects of bovine histone 1.

### Example 2

Determination of the cytotoxicity of H1° and H1.2 histones isolated from E. coli and the H1 histones from bovine thymus to the leukaemia cell line K562 and to PBMCs.

The PBMCs were isolated from Buffy Coats (layer of white cells which forms between the layer of red cells and the plasma when non-agglutinated blood is centrifuged or left to stand) of healthy donors using Ficoll Hypaque (Sigma, Deisenhofen, Germany), washed three times using PBS and resuspended in a density of 5×10⁵/ml in 10% FCS containing RPMI.

The K562 cells were cultured in RPMI with 10% FCS. The cells were harvested during logarithmic cell growth, washed once and stored in a density of 5×10⁵/ml.

For the cytotoxicity assays the PBMCs and the K562 cells were incubated in a concentration of 5×10⁴ cells/well in a 96-well flat-bottomed plate with and without different concentrations of histones.

The cytotoxicity was determined by measuring the propidium iodide incorporation using an EPICS XL flow cytometer (Coulter & Beckmann, Hamburg, Germany). At the end of the incubation period the cells were resuspended and 200 μl of the cell suspension was mixed with 200 μl of propidium iodide solution to give a final concentration of 2.5 μg per ml of propidium iodide (Sigma, Deisenhofen, Germany). The percentage fraction of cells which have incorporated propidium iodide from a total of 10,000 counted cells was determined directly after adding the propidium iodide solution and designated as the percentage toxicity.

After incubation for one hour, all three histone preparations, i.e. the histones H1° and H1.2 produced recombinantly in E. coli and the histone isolated from bovine thymus, induced a significant and dose-dependent toxicity to the leukaemia cell line K562 (FIGS. 2A, B, C, solid lines).

H1° exhibited a weak increase in cytotoxicity when the histone concentration was increased to 125 μg/ml and a stronger increase at up to 250 and 500 μg/ml histone. At this concentration 35% of the leukaemia cell lines died (FIG. 2A).

H1.2 and bovine histone 1 showed a strongly increasing toxicity in the range of 62 μg/ml to 125 μg/ml, in which 63% and 65% of the tumour cells died (FIGS. 2B, C). A further increase in the histone concentration to 500 μg per ml led to no increase (H1.2) or to only a slight (bovine histone 1) increase in cytotoxicity (see FIGS. 2B, C).

The cytotoxic effects on PBMCs were less pronounced (see FIGS. 2A, B, C, dashed lines). After incubation for one hour with 500 μg/ml histone H1°, 93.3% of the cells were still viable (see FIG. 2A). Under the same experimental conditions 87.4% or 91% respectively of the PBMCs survived incubation with recombinant H1.2 and bovine H1 preparation (see FIGS. 2B, C).

The time dependence of the histone cytotoxicity was measured at 250 μg/ml histone. The tumour cell cytotoxicity of all three histone preparations began directly after the beginning of incubation and reached its maximum during the first 30 minutes of incubation (see FIG. 3A). In the case of histone H1.2 and bovine histone H1, 52.8% or 56.3% respectively of the leukaemia cells were dead within the first 10 minutes. After 30 minutes the number of cells stained with propidium iodide increased to 74.5% and 78.6%, respectively. In the case of H1° a toxicity of 35.2% was observed within the first 10 minutes which merely increased by a further 11% after 30 minutes.

The toxicity of H1.2 and bovine histone for tumour cell lines remained approximately stable over a period of 24 hours whereas the toxicity of H1° decreased during the same period.

In contrast to the results for tumour cells, the human histones H1° and H1.2 and the bovine histone 1 showed no significant toxicity to PBMCs within the first hour of incubation (see FIG. 3B). After 4 and 24 hours, however, the toxicity increased to values which were comparable to the toxicity to K562 cells.

### DRAWINGS

FIG. 1: Purity of histones. Aliquots of recombinant H1° (A), H1.2 (B) and bovine histone 1 (C) were rechromatographed using analytical reversed-phase HPLC. 2 μg of each histone was analysed using SDS-PAGE on a 15% gel. The gels stained with Coomassie blue are shown. The main peaks in (c) are denoted by 1, 2 and 3 according to their retention time.

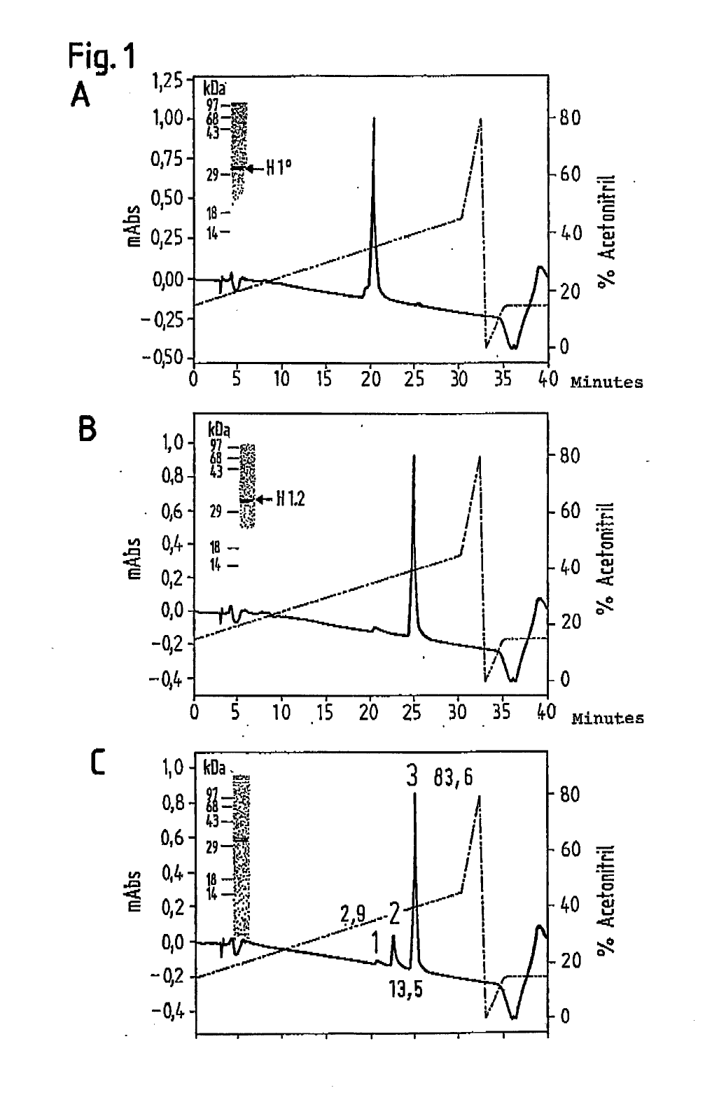

FIG. 2: Dose-dependent cytotoxicity of histones. The cytotoxicity of the recombinant histones H1° (A), H1.2 (B) and bovine histone H1 (C) to PBMCs and to the leukaemia cell line K562 (human chronic myeloid leukaemia; Lozzio et al. (1973) J. Natl. Cancer Inst. 50:535-538; Zozzio et al. (1975) Blood 45:321-334) was measured after 60 minutes by PI (propidium iodide) staining.

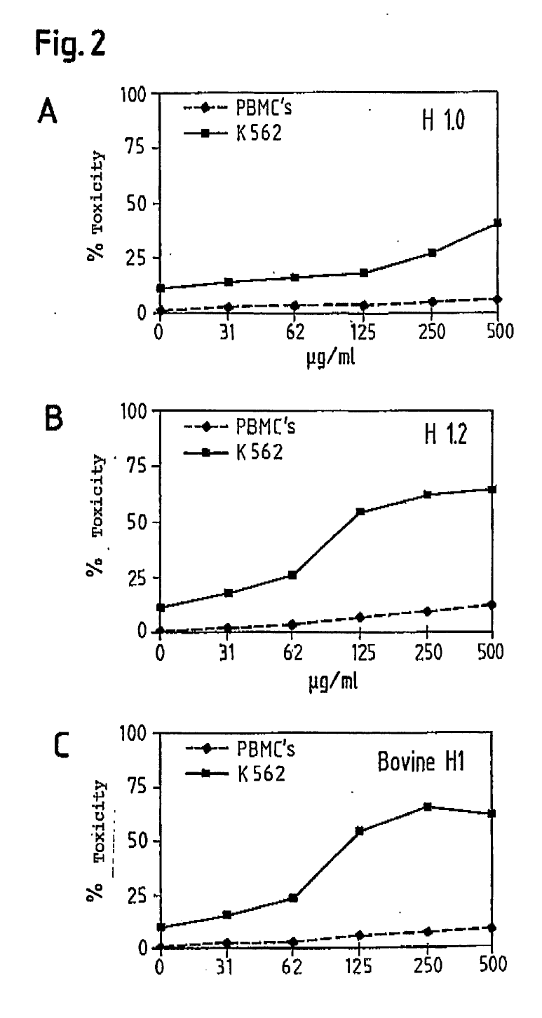

FIG. 3: Time dependence of the histone cytotoxicity. The leukaemia cell line K562 (a) and PBMCs (b) were incubated with 250 μg/ml of human recombinant H1° or H1.2 and bovine histone. The degree of toxicity was measured by PI staining.

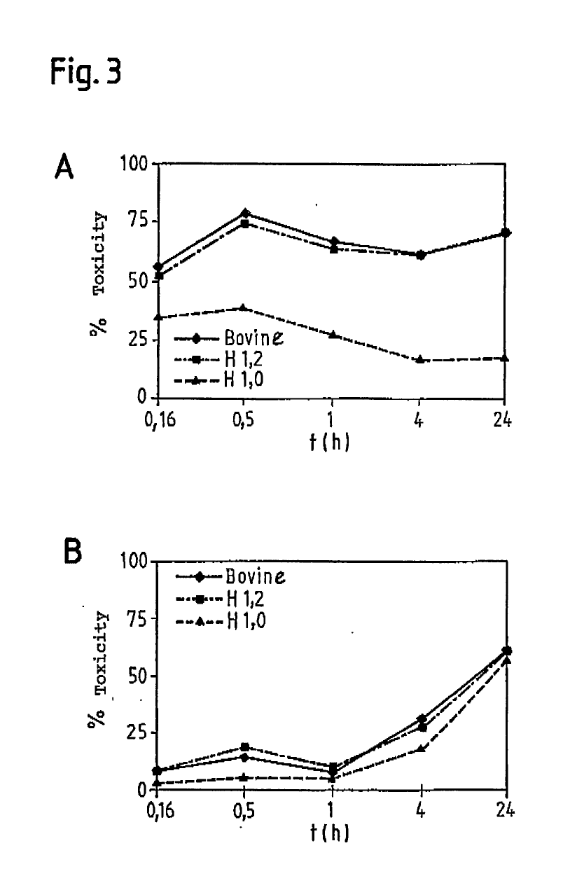

What is claimed is:

1. Recombinant human protein histone 1 or an active fragment thereof, produced in prokaryotic cells.

2. Protein according to claim 1, where the prokaryotic cell is E. coli.

3. Protein according to claim 1 or 2, where the histone 1 is H1° or H1.2.

4. Protein according to any of the preceding claims, where the histone 1 is H1.2.

5. Method of producing recombinant human protein histone 1 or an active fragment thereof, comprising the following steps:
a) expression of a nucleic acid sequence coding for human protein histone 1 or for an active fragment thereof in prokaryotic cells, and
b) extraction and purification of the recombinant human protein having th biological activity of a histone 1 or an active fragment thereof from the prokaryotic cells.

6. Method according to claim 5, where the prokaryotic cell is E. coli.

7. Method according to claim 5 or 6, where the histone 1 is H1° or H1.2.

8. Method according to one of claims 5 to 7, where the histone 1 is H1.2.

9. Use of recombinant human protein histone 1 or an active fragment thereof according to any of claims 1 to 4 for therapeutic purposes.

10. Use according to claim 9 for cancer therapy.

11. Use according to claim 9 or 10 for the therapy of carcinomas, melanomas, sarcomas, mesotheliomas and malignant diseases, in particular of malignant diseases of the lymphatic system, caused by malignant B- and T-cells, such as B-lymphoblastic lymphoma, myelogenic lymphoma or Burkitt-lymphoma.

12. Use according to claim 9 as antibiotic.

13. Use according to claim 9 for immunotherapy, in particular for the therapy of autoimmune diseases.

14. Use according to aim 9 for the therapy of endocrine disorders.

15. Use of recombinant human protein histone 1 or an active fragment thereof according to any of claims 1 to 4 as carrier for nucleic acids.

16. Use according to claim 15, wherein the nucleic acids serve as vaccine.

17. Use of recombinant human protein histone 1 or an active fragment thereof according to any of claims 1 to 4 in ex vivo treatment of cells with nucleic acids.

18. Use according to claim 17, where the recombinant human protein histone 1 or active fragment thereof are used as transfection adjuvants.


SEQUENCE LISTING (4 sequences)

SEQ ID NO: 1 - DNA, 39 bp, Artificial Sequence
  A primer
ggggggggatccatatgaccgagaattccacgtccgccc

SEQ ID NO: 2 - DNA, 39 bp, Artificial Sequence
  A primer
gggggggtcgactcacttcttcttgccggccctcttggc

SEQ ID NO: 3 - DNA, 37 bp, Artificial Sequence
  A primer
ggggggggatccatatgtccgagactgctcctgccgc

SEQ ID NO: 4 - DNA, 37 bp, Artificial Sequence
  A primer
gggggggtcgacctatttcttcttgggcgccgccttc